(19) United States
(12) Patent Application Publication
(10) Pub. No.: US 2004/0040060 A1
(43) Pub. Date: Feb. 26, 2004

(54) Novel plant seed mixtures

(76) Inventor: Gregory St. Clair, Hilliard, OH (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/462,241

(22) Filed: Jun. 16, 2003

Related U.S. Application Data
(60) Provisional application No. 60/403,956, filed on Aug. 16, 2002.

(51) Int. Cl. A01H 1/00; A01H 5/00; C12N 15/82

(52) U.S. Cl. 800/281; 800/312

(57) ABSTRACT

Plant seed compositions are disclosed that comprise a specialty or agronomic trait variety as a major component and one or more additional varieties as minor components. Each variety is phenotypically distinguishable from all other varieties in the seed mixture.

### CROSS-REFERENCE TO RELATED APPLICATION

This application claims priority from U.S. Provisional Application No. 60/403,956, filed Aug. 16, 2002, and titled “Novel Plant Seed Mixtures,” which is incorporated by reference in its entirety.

### TECHNICAL FIELD

This invention relates to plant seed compositions, more particularly, seed mixtures containing varieties that are phenotypically distinguishable from each other.

### BACKGROUND

Plants having novel and useful agronomic or specialty traits are increasingly used in production agriculture. Such plants sometimes require the application of chemicals, for example, to induce synthesis of a specialty component or to induce resistance to a pest. Unfortunately, plants having a particular agronomic or specialty trait often are not visually distinguishable from similar plants that lack the trait, especially during early stages of plant growth. This means there is a risk that the crops from both types of plants may be commingled at harvest time. If there is a necessity for chemical application, the chemical may be applied to sensitive plants, leading to increased expense and possibly complete loss of the sensitive plants. As varieties having a particular agronomic or specialty trait become more prevalent, the incidence of commingling or misapplication may decrease. However, as other different agronomic or specialty traits are developed, the risk of commingling or misapplication can once again increase.

### SUMMARY

The invention features an article of manufacture, comprising packaging material and plant seeds within the packaging material. The plant seeds can comprise about 80 to about 99.9 percent seeds of a first variety and about 0.1 to about 20 percent seeds of a second variety. Plants grown from seeds of the first variety exhibit a particular specialty or agronomic trait. Plants grown from seeds of the second variety may or may not exhibit the same specialty or agronomic trait. Plants of the first and second varieties are phenotypically distinguishable from each other. The plant seeds can be, e.g., Brassica seeds or soybean seeds. The first and second varieties can have different relative maturities.

Plants of the first variety can be phenotypically distinguishable from plants of the second variety by a difference in leaf color, leaf color, or leaf number. For example, plants of the first variety can have green trifoliate leaves, and plants of the second variety can have yellow trifoliate leaves. In some embodiments, plants of the first variety have green trifoliate leaves and the plants of the second variety have green variegated trifoliate leaves. In other embodiments, plants of the second variety have yellow pentafoliate leaves, or green pentafoliate leaves, or yellow heptafoliate leaves, or a fasciated stem. In some embodiments, the plant seeds can comprise about 0.1 to about 5 percent seeds of a third variety. The seeds of at least one of the varieties can have microparticles adhered thereto. The microparticles can be dual color microparticles. In some embodiments, seeds of at least one of the varieties has a seed coat color that differs from at least one other of the varieties, e.g., seeds of the first and third varieties have the same seed coat color and seeds of the second variety have a seed coat color that differs from the seed coat color of the first and third varieties.

The invention also features a composition comprising about 80 to about 99.9 percent seeds of a first variety, and about 0.1 to about 20 percent seeds of a second variety, wherein plants of the first variety exhibit a specialty or agronomic trait, and plants of the second variety do not exhibit the specialty or agronomic trait, and wherein plants of the first and second varieties are phenotypically distinguishable from each other. In some embodiments, the composition can comprise about 0.1 to about 5 percent seeds of a third variety.

The invention also features a method for producing a specialty soybean crop. The method comprises harvesting a crop grown from a group of crop plants comprising about 80 to about 99.9 percent seeds of a first variety and about 0.1 to about 20 percent seeds of a second variety. Plants of the first variety exhibit a specialty trait, and plants of the first and second varieties are phenotypically distinguishable from each other. In some embodiments, plants of the first and second varieties have different relative maturities. The first variety can belong to maturity group V and the second variety can belong to maturity group 0, I, II, or III. The first variety can belong to maturity group I and the second variety can belong to maturity group II, III, IV, or V.

The invention also features a method of making an article of manufacture. The method comprises packaging plant seeds within a packaging material. The plant seeds comprise about 80 to about 99.9 percent seeds of a first variety, and about 0.1 to about 20 percent seeds of a second variety. Plants of the first variety exhibit a specialty or agronomic trait and plants of the first and second varieties are phenotypically distinguishable from each other.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### DETAILED DESCRIPTION

The inventors have discovered novel compositions that comprise a mixture of seeds from two or more varieties of a single plant species. A first variety constitutes the majority of seed in the composition, and the remaining variety (or varieties) together constitute a minority of seed in the composition. Each of the varieties in the mixture can be distinguished from all other varieties in the composition on the basis of phenotypic markers. The first variety exhibits a particular specialty or agronomic trait, but is phenotypically distinguishable from the second variety. The second variety may or may not possess the particular specialty or agronomic trait, but is phenotypically distinguishable from the first variety. The second variety can have a different maturity than the first variety.

Such a seed composition is useful to farmers and agricultural companies, such as seed companies or farm supply companies. A group of plants grown from such a composition permits one to identify fields having a preselected specialty or agronomic trait, e.g., by visual inspection for the presence of the second variety. A group of plants grown from such a composition also can permit one to identify fields suitable for application of a chemical, when such a chemical is necessary for the specialty or agronomic trait.

Seed Compositions

A plant seed composition of the invention contains seeds of at least two varieties. The proportion of each variety in a composition is measured as the number of seeds of a particular variety divided by the total number of seeds in the composition, and can be formulated as desired to meet requirements based on geographic location, desired maturity and the like. The proportion of the first variety can be from about 80 percent to about 99.9 percent, e.g., 80%, 85%, 90%, 91%, 92%, 93%, 94%, 95%, 96%, 97%, 98%, or 99%. The proportion of the second variety can be from about 0.1 percent to about 20 percent, e.g., 0.5%, 1%, 2%, 3%, 4%, or 5%. If a third variety is present in the composition, the proportion of the third variety can be from about 0.1 percent to about 5 percent, e.g., 0.5%, 1%, 2%, 3%, 4%, or 5%. When large quantities of a seed composition are formulated, or when the same composition is formulated repeatedly, there may be some variation in the proportion of each variety due to sampling error. Sampling error is known from statistics. In the present invention, such sampling error typically is about ±5% of the expected proportion, e.g., 90%±4.5%, or 5%±0.25%. A seed composition can be formulated in a quantity of about 35 kilograms (kg) or more, about 100 kg or more, about 1,000 kg or more, about 10,000 kg or more, or about 50,000 kg or more.

Plants grown from seeds of the first variety exhibit a particular specialty or agronomic trait. Plants grown from seeds of the second variety may or may not exhibit the particular specialty or agronomic trait, but are phenotypically distinguishable from plants of the first variety on the basis of one or more phenotypic markers. Second variety plants also can have a different maturity than plants of the first variety.

For example, a seed composition of the invention can be made from two soybean varieties. A first soybean variety can constitute 99.5% of the seeds in the composition and exhibit decreased linolenic acid content in its harvested seeds, with green trifoliate leaves. A second soybean variety can constitute 0.5% of the seed in the composition and have typical commodity levels of linolenic acid in its harvested seeds, with yellow trifoliate leaves. Thus, plants of each of the varieties is phenotypically distinguishable from the other variety in the mixture.

Typically, a substantially uniform mixture of seeds of each of the varieties is conditioned and bagged in packaging material by means known in the art to form an article of manufacture. Such a bag of seed preferably has a package label accompanying the bag, e.g., a tag or label secured to the packaging material, a label printed on the packaging material or a label inserted within the bag. The package label indicates that the seeds therein are a mixture of varieties, e.g., three different varieties. The package label may indicate that plants grown from such seeds permit one to whether the plants possess a particular specialty or agronomic trait. The package label also may indicate that the seed mixture contained therein permits detection if seed saved from the harvested crop is replanted in a subsequent growing season.

In some embodiments, a plant seed composition further comprises additional varieties, e.g., about 0.1 to about 5 percent seeds of a third variety. Plants grown from seeds of the third variety are phenotypically distinguishable from all other varieties in the composition, e.g., the first and the second varieties. Plants of such additional varieties may or may not exhibit the same specialty or agronomic trait as do plants of the first variety.

Varieties in a seed composition of the invention typically have the same or very similar maturity, i.e., the same or very similar number of days from germination to crop seed maturation. In some embodiments, however, one or more varieties in a seed composition of the invention can have a different relative maturity compared to other varieties in the composition, i.e., the number of days from germination to mature seed for one variety in a composition is statistically significantly different from that of another variety in the composition. For example, the first variety in a composition of soybean seeds can be classified as belonging to the Group V relative maturity group, while the second variety can be classified in the Group I relative maturity group. As another example, the first variety in a composition of soybean seeds can be classified as belonging to the Group 0 relative maturity group, while the second variety can be classified in the Group II relative maturity group. The presence of varieties of different relative maturities in a seed composition can be useful when it is desired to reduce the amount of second variety seeds in the harvested crop. Relative maturity of a variety of a given crop species is classified by techniques known in the art.

A number of plant species are suitable for practicing the invention. Species that are naturally self-pollinated are particularly suitable, e.g., dicotyledonous species such as soybean, including natto and tofu soybeans, and rapeseed. Also suitable are vegetable crops or root crops such as potato, broccoli, peas, sweet corn, popcorn, tomato, beans (including kidney beans, lima beans, dry beans, green beans) and the like. Suitable plants include species from the genera Arachis, Asparagus, Alropa, Brassica, Citrus, Citrullus, Capsicum, Carthamus, Cocos, Coffea, Cucumis, Cucurbila, Daucus, Elacis, Fragaria, Glycine, Gossypium, Helianthius, Helerocallis, Lactuca, Linum, Lupinus, Lycopersicon, Malus, Manihot, Majorana, Medicago, Nicoliana, Olea, Persea, Phaseolus, Pistachia, Pisuni, Pyrus, Prun is, Raphanus, Ricinus, Senecio, Sinapis, Solanum, Theobromus, Trigonella, Vicia, Vitis, and Vigna. See, e.g., U.S. Pat. No. 6,274,793.

In some embodiments, a seed composition of the invention contains varieties of different plant species, e.g., the first plant variety is soybean and the second plant variety is rapeseed.

Agronomic and Specialty Traits

The first variety in a seed composition exhibits at least one specialty or agronomic trait. The minor varieties in seed composition optionally may exhibit the same trait(s) as the first variety. Specialty traits can be due to one or more naturally occurring variant genes, one or more transgenes, or a combination of naturally occurring variant gene(s) and transgene(s). Specialty traits include, for example, increased oil content, increased protein content, increased peroxidase content, and increased nutraceutical content, e.g., increased phytosterol content, increased tocopherol content, increased stanol content or increased vitamin content. Specialty traits also include, for example, a reduced content of an unwanted component in a harvested crop, e.g., phytic acid, soybean trypsin inhibitor, or lipoxygenase. Varieties possessing a gene(s) for a specialty trait have a statistically significant increase or decrease in the trait relative to a variety that does not possess or does not express those gene(s). Examples of plants exhibiting a specialty trait include soybeans having low linolenic acid and elevated stearic acid contents, elevated concentrations of both palmitic acid and stearic acid, low saturated fatty acid content, or reduced linolenic acid content. See, e.g., U.S. Pat. Nos. 5,714,668; 5,795,969; 5,750,845; 5,850,030; 5,530,183; 5,516,980; 6,171,640; and 6,423,886.

Agronomic traits include traits that primarily are of benefit to a seed company, a grower, or a grain processor. Agronomic traits include virus resistance, bacterial pathogen resistance, insect resistance and fungal resistance. See, e.g., U.S. Pat. Nos. 5,569,823; 5,304,730; 5,495,071; 6,329,504; and 6,337,431. Varieties possessing a gene(s) for an agronomic trait have a statistically significant increase or decrease in the trait relative to a variety that does not possess or does not express those gene(s). A difference between two varieties in a trait is considered to be statistically significantly different at a p≦0.05 with an appropriate parametric or non-parametric statistic. A suitable parametric or non-parametric statistic includes a Chi-square test, a Student's t-test, a Mann-Whitney test, or an F-test. In some embodiments, a difference is considered statistically significant at p<0.01, p<0.005, or p<0.001.

In some instances, expression of a specialty or agronomic trait is inducible. See, e.g., U.S. Pat. No. 5,767,369 to Ryals et al. and U.S. Pat. No. 5,977,441 to Oliver et al. for a discussion of techniques by which a specialty or agronomic trait can be made inducible.

Phenotypic Markers

Each variety in a seed composition possesses a phenotypic marker other than herbicide resistance that is associated with that variety, and which distinguishes that variety from other varieties in the composition, i.e., characterizes that variety relative to other varieties in the composition. Phenotypic markers permit each of the varieties present in a seed composition to be distinguished during vegetative stages of growth. Generally, phenotypic markers permit varieties to be identified by visual inspection, although it is contemplated that phenotypic markers such as isozyme polymorphisms or nucleic acid polymorphisms can be used. In some embodiments, a phenotypic marker is a difference between two or more varieties in visible, near-infrared, or infrared radiation characteristics. Such a difference can permit varieties to be distinguished by remote sensing, e.g., satellite imaging. See, e.g., U.S. Pat. Nos. 5,764,819, 6,118,885, 6,160,902, 6,366,681 and 5,253,302. Phenotypic markers should be sufficiently stable to allow varieties to be distinguished despite variation in disease pressure, insect pressure, environment, or agronomic practices, recognizing that a phenotypic marker that is sufficiently stable in one geographic region may not be sufficiently stable in a different geographic region. Typically, a single marker is used to phenotypically distinguish each variety in the composition from all other varieties. It will be appreciated, however, that a variety can have two or more phenotypic differences from any other variety in the composition.

A number of phenotypic differences can be used to distinguish different varieties. For example, in soybean, one can incorporate phenotypic markers such as: leaf number (3, 5, 7, 9, or 11), leaf color (yellow, light green, medium green, or green), leaf color uniformity (uniform or variegated), cotyledon color (yellow or green), leaflet shape (rugose or lanceolate), or plant habit (determinate or indeterminate). Additional phenotypic markers include, without limitation, fasciated stem and dense pubescence. Table 1 summarizes some possible phenotypic combinations that can be used to distinguish between different soybean varieties. The numeral indicates the number of leaves per petiole present on the plant, e.g., 3L indicates three leaves, 5L indicates five leaves, and 7L indicates seven leaves. The letter “Y” indicates that the colors of the leaves are yellow.

TABLE 1
Herbicide Sensitive and Resistant
Combinations of Soybean Phenotypes

| Herbicide Sensitive Combinations | Herbicide Resistant Combinations |
|---|---|
| 3LY combinations | 3LY combinations |
| 3LY yellow seed coat | 3LY yellow seed coat |
| 3LY brown seed coat | 3LY brown seed coat |
| 3LY black seed coat | 3LY black seed coar |
| 3LY green seed coat | 3LY green seed coat |
| 3LY Lanceolate leaves | 3LY Lanceolate leaves |
| 3L Variegated leaves | 3L Variegated leaves |
| 3L Lanceolate and Variegated | 3L Lanceolate and Variegated |
| leaves | leaves |
| 3LY rugose yellow seed coat | 3LY rugose yellow seed coat |
| 3LY rugose brown seed coat | 3LY rugose brown seed coat |
| 3LY rugose black seed coat | 3LY rugose black seed coat |
| 3LY rugose green seed coat | 3LY rugose green seed coat |
| 3LY rugose Lanceolate leaves | 3LY rugose Lanceolate leaves |
| 3L rugose Variegated leaves | 3L rugose Variegated leaves |
| 3L rugose Lanceolate, | 3L rugose Lanceolate, |
| Variegated leaves | Variegated leaves |
| 5LY combinations | 5LY combinations |
| 5LY yellow seed coat | 5LY yellow seed coat |
| 5LY brown seed coat | 5LY brown seed coat |
| 5LY black seed coat | 5LY black seed coat |
| 5LY green seed coat | 5LY green seed coat |
| 5LY Lanceolate leaves | 5LY Lanceolate leaves |
| 5L Variegated leaves | 5L Variegated leaves |
| 5L Lanceolate and Variegated | 5L Lanceolate and Variegated |
| leaves | leaves |
| 5LY rugose yellow seed coat | 5LY rugose yellow seed coat |
| 5LY rugose brown seed coat | 5LY rugose brown seed coat |
| SLY rugose black seed coat | 5LY rugose black seed coat |
| 5LY rugose green seed coat | 5LY rugose green seed coat |
| 5LY rugose Lanceolate leaves | 5LY rugose Lanceolate leaves |
| 5L rugose Variegated leaves | 5L rugose Variegated leaves |
| 5L rugose Lanceolate, | 5L rugose Lanceolate, |
| Variegated leaves | Variegated leaves |
| 7LY combinations | 7LY combinations |
| 7LY yellow seed coat | 7LY yellow seed coat |
| 7LY brown seed coat | 7LY brown seed coat |
| 7LY black seed coat | 7LY black seed coat |
| 7LY green seed coat | 7LY green seed coat |
| 7LY Lanceolate leaves | 7LY Lanceolate leaves |
| 7L Variegated leaves | 7L Variegated leaves |
| 7L Lanceolate and Variegated | 7L Lanceolate and Variegated |
| leaves | leaves |
| 7LY rugose yellow seed coat | 7LY rugose yellow seed coat |
| 7LY rugose brown seed coat | 7LY rugose brown seed coat |
| 7LY rugose black seed coat | 7LY rugose black seed coat |
| 7LY rugose green seed coat | 7LY rugose green seed coat |
| 7LY rugose Lanceolate leaves | 7LY rugose Lanceolate leaves |
| 7L rugose Variegated leaves | 7L rugose Variegated leaves |
| 7L rugose Lanceolate, | 7L rugose Lanceolate, |
| Variegated leaves | Variegated leaves |

A phenotypic marker typically is genetically determined by nuclear or cytoplasmic inheritance. A phenotypic marker can be due to a naturally occurring variant or can be conferred by a transgene. It is contemplated that a trait conferred by a plant virus can also be useful. It will be appreciated that some phenotypic markers may have some similarity to symptoms observed due to disease pressure, nutrient deficiency, or weather. For example, manganese deficiency may be observed on soybean plants in some geographic areas, e.g., well-drained neutral and alkaline soils with a high pH. Symptoms characteristic of manganese deficiency include yellow leaves. See Compendium of Soybean Diseases, J. B. Sinclair, ed., 2^{nd} Edition, p. 79, The American Phytopathological Society (1982). The practitioner will recognize and take into account the possibility of such similarities when formulating seed compositions of the invention for a given geographic area.

Herbicide Resistance

In some embodiments, one or more of the plant varieties in a seed composition of the invention exhibit resistance to a herbicide. A number of genes are available, both transgenic and non-transgenic, that confer herbicide resistance. Herbicide resistance is also sometimes referred to as herbicide tolerance. Genes conferring resistance to a herbicide that inhibits the growing point or meristem, such as an imidazalinone or a sulfonylurea can be suitable. Exemplary genes in this category code for mutant ALS and AHAS enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS) resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Genes for resistance to glyphosate (sold under the trade name Roundup®) are also suitable. See, for example, U.S. Pat. No. 4,940,835 and U.S. Pat. No. 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Genes for resistance to phosphono compounds such as glufosinate ammonium or phosphinothricin, and pyridinoxy or phenoxy propionic acids and cyclohexones are also suitable. See European application No. 0 242 246. See also, U.S. Pat. Nos. 5,879,903, 5,276,268 and 5,561,236.

Other suitable herbicides include those that inhibit photosynthesis, such as a triazine and a benzonitrile (nitrilase). See U.S. Pat. No. 4,810,648. Other suitable herbicides include 2,2-dichloropropionic acid, sethoxydim, haloxyfop, imidazolinone herbicides, sulfonylurea herbicides, triazolopyrimidine herbicides, s-triazine herbicides and bromoxynil. Also suitable are herbicides that confer resistance to a protox enzyme. See, e.g., U.S. Patent Application No. 20010016956, and U.S. Pat. No. 6,084,155.

For example, soybean (Glycine max) is an important and valuable field crop. Soybean breeders develop stable, high yielding soybean varieties that are agronomically sound. To this end, soybean breeders have developed transgenic and non-transgenic varieties that possess resistance to certain herbicides. Exemplary glyphosate resistant soybean varieties include those disclosed in U.S. Pat. Nos. 6,353,157; 6,350,938; 6,346,657; 6,323,398 and 6,313,380. Additional herbicide resistant soybean varieties can be developed from a transgenic soybean line designated 40-3, which carries a gene conferring resistance to Roundup® herbicide.

Herbicide resistant wheat varieties have been developed. Exemplary herbicide resistant wheat varieties include those disclosed in U.S. Pat. No. 6,339,184. Herbicide resistant canola varieties are discussed in U.S. Pat. No. 5,545,821. Exemplary herbicide resistant rice varieties include those disclosed in U.S. Pat. No. 5,952,553.

Additional Markers

In some embodiments, seeds of the second variety in a composition of the invention have a seed coat color that differs from other varieties in the composition. A different seed coat color can be used to mark a composition as having a specialty or agronomic trait. Furthermore, the resulting harvested crop can contain seeds having the seed coat color of the second variety, thus marking the crop as having a specialty or agronomic trait. In addition, a farmer sometimes saves a portion of the harvested crop for use as seed to be replanting in subsequent growing seasons. Saved seeds are often conditioned and packaged by a seed conditioner before such replanting. The presence of seeds having different seed coat colors permits a seed conditioner to distinguish between specialty or agronomic trait seed and commodity seed, and thus condition specialty or agronomic trait of seed separately from other types of seed.

In some embodiments, each variety in a seed composition has a different seed coat color. For example, a composition can have 99.9% seeds of a first variety having a tan seed coat color, 0.075% seeds of a second variety having a green seed coat color, and 0.025% seeds of a third variety having a black seed coat color. Another suitable seed coat color is brown. In other embodiments, seeds of the second and third varieties in a composition have the same seed coat color, which seed coat color differs from the seed coat color of the first variety in the composition. For example, a first variety can have a tan seed coat color, whereas the second and third varieties have a black seed coat color. In other embodiments, seeds of the first and second varieties in a composition have the same seed coat color, which seed coat color differs from the seed coat color of the third variety in the composition. In other embodiments, seeds of the first and third varieties in a composition have the same seed coat color, which seed coat color differs from the seed coat color of the second variety in the composition.

A seed composition having varieties with different seed coat colors can be useful to farmers and agricultural companies, since it permits identification of whether harvested seeds contain a particular specialty or agronomic trait. For example, a seed composition containing seeds of a first variety having a tan seed coat color, seeds of a second variety having a green seed coat color, and seeds of a third variety having a black seed coat color, will result in a harvested crop that contains tan seeds and black seeds, thereby marking the crop as containing the particular specialty or agronomic trait(s) carried by that seed composition. Thus, in another aspect, the invention features a method of marking a harvested crop. The method involves growing a seed composition of the invention, and harvesting a crop from plants that result from growing the composition.

In some embodiments, microparticles are used to mark one or more varieties in a seed composition. Microparticles adhered to individual seeds of a particular variety permit ready identification of that variety. A specific series of microparticle types can be used, each series adhered to seeds of a particular variety. Alternatively, a single type of microparticles can be used, such a type adhered to seeds of only one of the varieties in a seed composition. As another alternative, microparticles can be used to mark an individual article of manufacture, e.g., by adhering microparticles to packaging material or to a package label accompanying the article. In the case of a plurality of articles, microparticles can be combined with or adhered to a packaging or shipping container that contains the plurality of articles.

Microparticles can be combined with a binder, for instance an adhesive or coating formulation. Suitable binder materials are known. The resulting particle/adhesive mixture can, for example, then be applied to the surface of individual seeds for identification purposes.

A marked seed(s) can be observed to determine the presence or absence of microparticles. If the microparticles are visible to the naked eye, the examination may be performed without additional equipment. For microparticles that are not easily visualized by the naked eye, equipment such as a light microscope or a magnifying glass may be used. Typically, microparticles can be examined using a common 40× or 100× microscope.

The presence or absence of specific microparticles can be detected and recorded. An individual can perform the detection and recordation manually. Alternately, an automated system, e.g., a computerized system, can perform detection and recordation.

Microparticles having a single colored layer can be used, recognizing that certain colors may not be suitable for particular seed coat colors. For example, a tan microparticle would render identification difficult if the marked variety had a tan seed coat color. Microparticles having two colored layers can be used. Dual layer microparticles can often provide a sufficient diversity of color combinations. Alternatively, a 5-layered particle can be used. If desired, microparticles can include visual enhancers. Suitable visual enhancers include, without limitation, pearlescent colorant, glitter, metal flake pigments and glass microspheres. Visual enhancers can provide microparticles with a higher localized reflectance and a more characteristic appearance, making the colored layer(s) of a microparticle are more easily distinguishable. Visual enhancers can also further differentiate color layers of one type of microparticle from another type of microparticle. For example, a visual enhancer can be added to distinguish one secondary color (i.e., orange, green, and purple) from another secondary color.

As an alternative to visually distinguishable characteristics, the layer(s) of different types of microparticles may be distinguished by machine-readable characteristics. Machine-readable characteristics can include magnetic characteristics, infrared or ultraviolet absorption characteristics, infrared or ultraviolet reflection characteristics, or fluorescence or visible light transmission characteristics.

Methods

In another aspect, the invention features a method for distinguishing between older and newer varieties of a particular specialty or agronomic trait. A seed composition of the invention can permit farmers and seed companies to distinguish between an older specialty or agronomic trait variety and a newer specialty or agronomic trait variety in a geographic region, i.e., fields planted with a specialty or agronomic trait variety that has been on the market for a period of time and nearby fields planted with a variety that has recently been introduced for sale and has the same specialty or agronomic trait. The older variety can be formulated during phase-out as the major proportion in a seed composition that contains a minor variety, while the newer variety can be formulated as the only seed in a composition. Alternatively, the older variety can be sold as the only seed in a composition, while the newer variety can be formulated during phase-in as the major proportion in a seed composition that contains a second variety.

As another alternative, a newer variety can be formulated during phase-in as the major portion of a seed composition that contains second and third varieties, while an older variety can be sold as the major portion in a seed composition that contains a minor portion of a fourth variety. The fourth variety is phenotypically distinguishable from the second and third varieties.

As another alternative, a variety exhibiting a specialty or agronomic trait can be formulated as the major proportion in a seed composition that contains an initial second variety, while a variety exhibiting a second, different specialty or agronomic trait can be formulated as the major portion in a seed composition that contains a minor portion of a third variety. The second variety is phenotypically distinguishable from all other varieties in the two compositions. The third variety is phenotypically distinguishable from all other varieties in the two compositions. The ratios of the major and minor varieties in a seed composition can be formulated as described herein.

It is known that planting seed is sometimes not sold in the year following its production. It is also known that the germination percentage of unplanted seed declines over time. The present invention allows an estimation of the year in which planting seed was intended to be sold, using seed compositions having one or more minor varieties. A seed composition of the invention can be formulated for a given geographic area with first and second varieties that are phenotypically distinguishable from each other on the basis on an initial phenotypic difference(s). In a subsequent growing season, a second seed composition of the invention is formulated for the same geographic area so that the first and second varieties are phenotypically distinguishable from each other on the basis of another, different phenotypic difference(s). The subsequent growing season can be the next growing season, two growing seasons later, or three or more growing seasons later. As an example, the first and second varieties in an initial soybean seed composition can have 3LG and 3LY phenotypes, respectively. The first variety exhibits a specialty trait. In a subsequent growing season, the first and second varieties in a second soybean seed composition can have 5LG and 5L variegated phenotypes, respectively. The first variety exhibits the same specialty trait.

Seed compositions having different phenotypic markers in different growing seasons can be useful to farmers and agricultural companies, since it permits an approximate date of production of a seed composition to be assigned to a group of plants in a field. That is, an approximate date can be assigned as to the growing season in which the seed composition was intended to be planted.

The invention is further described in the following examples, which do not limit the scope of the invention described in the claims.

### EXAMPLES

### Example 1

### Soybean Varieties

Soybean cultivars are bred using standard breeding practices to create varieties having the combinations of phenotypic markers described in Table 1. A yellow leaf phenotypic marker can be incorporated from, for example, the T135 strain, available from the U.S. Department of Agriculture (USDA) soybean germplasm collection. The T135 strain carries the y9 allele, which confers a bright greenish yellow color to leaves. A heptafoliate phenotypic marker can be incorporated from, for example, the T255 strain, available from the USDA. The T255 strain carries the lf2 allele, which confers a 7-foliate phenotype to leaves. A variegated leaf phenotypic marker can be incorporated from, for example, the T312 strain, available from the USDA. The T312 strain carries the v2 allele, which confers a variegated phenotype to leaves. A narrow, rugose leaflet phenotypic marker can be incorporated from, for example, the T313 strain, available from the USDA. The T313 strain carries the lnr allele, which confers a rugose phenotype to leaves.

Some of the criteria used to selecting cultivars include: seed yield, lodging resistance, emergence, disease resistance, maturity, late season plant intactness, plant height and shattering resistance. Breeders seed is entered in yield tests at several locations in the Midwest United States.

Cultivars typically show uniformity and stability, as described in the following variety description information. It is self-pollinated a Sufficient number of generations with careful attention to uniformity of plant type. The variety is increased with continued observation for uniformity.

Maturity Date. Plants are considered mature when 95% of the pods have reached their mature color. The number of days is either calculated from August 31 or from the planting date.

Seed Yield (Bushels/Acre). The yield in bushels/acre is the actual yield of the grain at harvest.

Lodging Resistance. Lodging is rated on a scale of 1 to 5. A score of 1 indicates erect plants. A score of 2.5 indicates plants are leaning at a 45° angle in relation to the ground and a score of 5 indicates plants are laying on the ground.

Phytophthora Tolerance/resistance. Phytophthora root rot tolerance is rated on a scale of 1 to 5, with a score of 1 indicating the best or highest tolerance, and a score of 5 indicating no tolerance to Phytophthora. Plants can be phytophthora resistant or sensitive, and those that are phytophthora resistant have nucleic acid resistant genes such as, for example, Rps1k and Rps1c.

Emergence. This score indicates the ability of the seed to emerge when planted 3″ deep in sand and with a controlled temperature of 25° C. The number of plants that emerge each day is counted. Based on this data, each genotype is given a 1 to 5 score based on its rate of emergence and percent of emergence. A score of 1 indicates an excellent rate and percent of emergence, an intermediate score of 2.5 indicates average ratings and a 5 score indicates a very poor rate and percent of emergence.

Iron-Deficiency Chlorosis. Plants are scored 1 to 5 based on visual observations. A score of 1 means no stunting of the plants or yellowing of the leaves and a score of 5 indicates the plants are dead or dying caused by iron-deficiency chlorosis, a score of 2.5 means plants have intermediate health with some leaf yellowing.

Brown Stem Rot. This is a visual disease score from 1 to 5 comparing all genotypes in a given test. The score is based on leaf symptoms of yellowing and necrosis caused by brown stem rot. A score of 1 indicates no symptoms. Visual scores range to a score of 5, which indicates severe symptoms of leaf yellowing and necrosis.

Shattering. The amount of pod dehiscence prior to harvest. Pod dehiscence involves seeds falling from the pods to the soil. This is a visual score from 1 to 5 comparing all genotypes within a given test. A score of 1 means pods have not opened and no seeds have fallen out. A score of 2.5 indicates approximately 50% of the pods have opened, with seeds falling to the ground and a score of 5 indicates 100% of the pods are opened.

Plant Height. Plant height is taken from the top of soil to top node of the plant and is measured in inches.

Seed Protein Peroxidase Activity. Seed protein peroxidase activity is defined as a chemical taxonomic technique to separate cultivars based on the presence or absence of the peroxidase enzyme in the seed coat. Generally, there are two types of soybean cultivars, those having high peroxidase activity (dark red color) and those having low peroxidase activity (no color).

Quantitative Trait Loci (QTL). Quantitative trait loci (QTL) refer to genetic loci that control to some degree numerically representable traits that are usually continuously distributed.

A number of embodiments of the invention have been described. Nevertheless, it will be understood that various modifications may be made without departing from the spirit and scope of the invention. Accordingly, other embodiments are within the scope of the following claims.

### REFERENCES

U.S. Pat. No. 5,004,864 U.S. Pat. No. 5,706,603

U.S. Pat. No. 5,704,160 U.S. Pat. No. 5,994,621

U.S. Pat. No. 5,859,349

What is claimed is:

1. An article of manufacture, comprising:
(a) packaging material; and
(b) plant seeds within said packaging material, said plant seeds comprising about 80 to about 99.9 percent seeds of a first variety, and about 0.1 to about 20 percent seeds of a second variety, wherein plants of said first variety exhibit a specialty or agronomic trait, and wherein plants of said first and second varieties are phenotypically distinguishable from each other.

2. The article of claim 1, wherein said plants of said first and said second variety have different relative maturities.

3. The article of claim 1, wherein said plants of said second variety exhibit said specialty or agronomic trait.

4. The article of claim 1, further comprising about 0.1 to about 5 percent seeds of a third variety, plants of said third variety being phenotypically distinguishable from said plants of said first and said second varieties.

5. The article of claim 1, wherein said plant seeds are soybean seeds and said trait is a specialty trait.

6. The article of claim 5, wherein said specialty trait is increased oil content.

7. The article of claim 5, wherein said specialty trait is increased protein content.

8. The article of claim 5, wherein said specialty trait is increased phytosterol content.

9. The article of claim 5, wherein said specialty trait is increased stanol content.

10. The article of claim 5, wherein said plants of said first variety exhibit resistance to glyphosate.

11. The article of claim 5, wherein said plants of said first variety exhibit resistance to phosphinothricill.

12. The article of claim 5, wherein said plants of said first and said second varieties are phenotypically distinguishable from each other on the basis of leaf color.

13. The article of claim 5, wherein said plants of said first and said second varieties are phenotypically distinguishable from each other on the basis of the number of leaves per petiole.

14. The article of claim 5, wherein said seeds of said first variety have a different seed coat color that said seeds of said second variety.

15. The article of claim 1, wherein seeds of at least one of said varieties has microparticles adhered thereto.

16. The article of claim 15, wherein said microparticles are dual color microparticles.

17. A method for producing a soybean crop comprising the steps of:
a) harvesting a crop grown from a group of crop plants comprising about 80 to about 99.9 percent seeds of a first variety and about 0.1 to about 20 percent seeds of a second variety, wherein plants of said first variety exhibit a specialty trait, plants of said first and second varieties are phenotypically distinguishable from each other.

18. The method of claim 17, wherein the relative maturity of said plants of said first variety is different from the relative maturity of said plants of said second variety.

19. The method of claim 18, wherein said first variety belongs to maturity group V and said second variety belongs to maturity group 0, I, II, or III.

20. The method of claim 18, wherein said first variety belongs to maturity group I and said second variety belongs to maturity group II, III, IV, or V.

21. The method of claim 17, wherein said specialty trait is increased oil content.

22. The method of claim 17, wherein said specialty trait is increased protein content.

23. The method of claim 17, wherein said specialty trait is increased phytosterol content.

24. The method of claim 17, wherein said specialty trait is increased stanol content.

25. The method of claim 17, wherein said plants of said second variety exhibit said specialty trait.

26. The method of claim 17, wherein said plants of said first variety exhibit resistance to glyphosate.

27. The method of claim 17, wherein said plants of said first variety exhibit resistance to phosphinothricin.

28. The method of claim 17, wherein said plants of said first and said second varieties are phenotypically distinguishable from each other on the basis of leaf color.

29. The method of claim 17, wherein said plants of said first and said second varieties are phenotypically distinguishable from each other on the basis of the number of leaves per petiole.

30. The method of claim 17, further comprising about 0.1 to about 5 percent seeds of a third variety, plants of said third variety being phenotypically distinguishable from said plants of said first and said second varieties.

31. A method of making an article of manufacture, comprising:
packaging plant seeds within a packaging material, said plant seeds comprising about 80 to about 99.9 percent seeds of a first variety, and about 0.1 to about 20 percent seeds of a second variety, wherein plants of said first variety exhibit a specialty or agronomic trait, and wherein plants of said first and second varieties are phenotypically distinguishable from each other.
